(19) United States
(12) Patent Application Publication
(10) Pub. No.: US 2021/0378519 A1
(43) Pub. Date: Dec. 9, 2021

(54) A BODY TEMPERATURE MONITORING SYSTEM

(71) Applicant: YILDIZ TEKNIK UNIVERSITESI, Besiktas/Istanbul (TR)

(72) Inventors: Fatma Aydin AKGUN, Davutpasa Kampusu, Esenler/Istanbul (TR); Giray SOYTURK, Kadikoy/Istanbul (TR); Muhtalip DEDE, Kartal/Istanbul (TR)

(73) Assignee: YILDIZ TEKNIK UNIVERSITESI, Besiktas/Istanbul (TR)

(21) Appl. No.: 17/284,114

(22) PCT Filed: May 23, 2020
(86) PCT No.: PCT/TR2020/050452

§ 371 Date: Apr. 9, 2021

(30) Foreign Application Priority Data
May 23, 2019 (TR) 2019/07825
May 22, 2020 (TR) 2020/08023

(51) Int. Cl. A61B 5/01; A61B 5/00; A61B 5/1171

(52) U.S. Cl. CPC A61B 5/01; A61B 5/0008; A61B 5/1176; A61B 2562/0233; A61B 2562/0271; A61B 2562/029; A61B 5/746

(57) ABSTRACT

The present invention is a temperature monitoring system for providing monitoring of the temperature of an individual (500) in a contactless manner, Accordingly, the subject matter temperature monitoring system is characterized in that a thermal sensor (110) is provided which is configured to generate temperature measurements in matrix form related to the area where it is directed, a distance sensor (140) is provided which is configured to face towards the area where said thermal sensor (110) is directed and to generate distance measurement in accordance with the distance of an individual (500), which exists in this area, with respect to said distance sensor (140), a processor unit (150) is provided which is connected to said thermal sensor (110) and to said distance sensor (140) in a manner receiving the temperature measurements and the distance measurements in matrix form as input, a communication unit (160) is provided which is connected to said processor unit (150) and which provides communication of the processor unit (150) with a server (200), said processor unit (150) is configured to send the temperature measurements and the distance measurements to said server (200), said server (200) is configured to select at least one of the temperature measurements in matrix form and to form an individual (500) body temperature value by arranging the selected temperature measurement with respect to the distance measurement

### TECHNICAL FIELD

The present invention relates to a temperature monitoring system for providing monitoring of the temperature of an individual in a contactless manner.

### PRIOR ART

High and low body temperature values may lead to permanent damages in the health of individuals. As a result of not being able to control body temperatures, diseases like seizure, paralysis, etc. can be faced. Thus, it is substantially important that body temperatures of individuals, who are in critical condition, like patient, baby, etc. must be continuously monitored. In cases depending on abnormalities in body temperature of a person, emergency action prevents temporary or permanent health problems of the person. In the present art, there are various solutions, which are integrated to the body for monitoring the change in body temperature. However, devices which are integrated to the body delimit the movement of said individuals and lead to discomfort. Moreover, these devices must be monitored by persons who look after the patient.

One of the solutions integrated to the body is thermometer. In traditional thermometers, it is difficult to provide continuous measurement and a person is needed who makes and controls the measurement. Moreover, there is the risk of contact of the harmful chemical substances to the skin of the patient or baby in case of a failure. Besides, digital temperature sensors which function by means of contact and digital temperature sensors which function in an infrared manner are also known in the art. However, the temperature sensor which functions by means of contact deteriorates the comfort of the patient. The temperature sensor, which realizes contactless measurement, must be directed to a patient by an operator and it must be ensured that correct measurement is taken. A continuous monitoring cannot be provided.

As a result, because of all of the abovementioned problems, an improvement is required in the related technical field.

### BRIEF DESCRIPTION OF THE INVENTION

The present invention relates to a temperature monitoring system, for eliminating the above mentioned disadvantages and for bringing new advantages to the related technical field.

An object of the present invention is to provide a temperature monitoring system which provides monitoring body temperatures of individuals like patient, baby, etc. in a contactless and remote manner.

Another object of the present invention is to provide a temperature monitoring system where the measurement precision is increased.

Another object of the present invention is to provide a temperature monitoring system which provides monitoring body temperature continuously and which increases elimination speed of emergent conditions.

In order to realize the abovementioned objects and the objects which are to be deducted from the detailed description below, the present invention is a temperature monitoring system for providing monitoring of temperature of an individual in a contactless manner. Accordingly, the subject matter temperature monitoring system is characterized in that a thermal sensor is provided which is configured to generate temperature measurements in matrix form related to the area where it is directed, a distance sensor is provided which is configured to face towards the area where said thermal sensor is directed and to generate distance measurement in accordance with the distance of an individual, which exists in this area, with respect to said distance sensor, a processor unit is provided which is connected to said thermal sensor and to said distance sensor in a manner receiving the temperature measurements and the distance measurements in matrix form as input, a communication unit is provided which is connected to said processor unit and which provides communication of the processor unit with a server, said processor unit is configured to send the temperature measurements and the distance measurements to said server, said server is configured to select at least one of the temperature measurements in matrix form and to form an individual body temperature value by arranging the selected temperature measurement with respect to the distance measurement. Thus, temperature monitoring can be realized in a remote and contactless manner where precision is increased.

In a preferred embodiment of the present invention, the server is configured to send warning message to at least one mobile device in case the formed individual body temperature value exceeds at least one predetermined first threshold value or in case the formed individual body temperature value is lower than at least one predetermined second threshold value. Thus, in case of abnormality, information can be obtained instantaneously and instantaneous intervention can be provided, and permanent, temporary health damage of the individual is prevented.

In another preferred embodiment of the present invention, a humidity sensor is provided which is connected to the processor unit and configured to realize humidity measurements in the medium where the individual exists, and the processor unit is configured to send the humidity measurements to the server, and the server is configured to form the body temperature of the individual by arranging the selected temperature measurement. Thus, body temperature measurement can be provided with increased precision.

In another preferred embodiment of the present invention, a medium temperature sensor is provided which is connected to the processor unit and configured to realize medium temperature measurements in the medium where the individual exists, and the processor unit is configured to send the medium temperature measurements to the server and the server is configured to form the body temperature value of the individual by arranging the selected temperature measurement by taking into account the medium temperature measurements.

In another preferred embodiment of the present invention, the server is arranged to select the biggest one among the temperature measurements in matrix form.

In another preferred embodiment of the present invention, the server is configured to send a message, indicating that there is an excessively hot object in the medium, to the mobile device in case the formed individual body temperature value exceeds a predetermined third threshold value and/or to send a message indicating that there is an excessively cold object in the medium in case the individual body temperature value is lower than a predetermined fourth threshold value. Thus, warning is formed for detecting and diverging cold and hot objects which are at danger level and which exist in the vicinity of the individual.

In another preferred embodiment of the present invention, the server is configured to store the formed individual body temperatures in a first database.

In another preferred embodiment of the present invention, the server is configured to store in a first database the humidity measurement, medium temperature measurement, selected temperature measurement and individual body temperature values and to realize the next individual body temperature formation processes by taking into account this database. Thus, the precision of determination of the body temperature is increased, in other words, the precision of calibration is increased by means of machine learning.

In another preferred embodiment of the present invention, a light sensor is provided which is configured to generate a signal according to the light received from the area where it is directed and to send this to the processor unit for being transferred to the server, and said server is configured to determine that the light sensor is directed to a human in case it is detected that the signals, received from said light sensor, is compliant to the predetermined criteria.

In another preferred embodiment of the present invention, the server is configured to generate a warning signal in case it is determined that the light sensor is not directed to a human. Thus, the erroneous measurements, which will occur in cases where the sensors are not directed to a human, are reduced.

In another preferred embodiment of the present invention, said light sensor is at least one of the thermal camera, color sensor, face recognition system and skin recognition system.

### BRIEF DESCRIPTION OF THE FIGURES

FIG. 1 is a representative view of the temperature monitoring system.

### DETAILED DESCRIPTION OF THE INVENTION

In this detailed description, the subject matter is explained with references to examples without forming any restrictive effect only in order to make the subject more understandable.

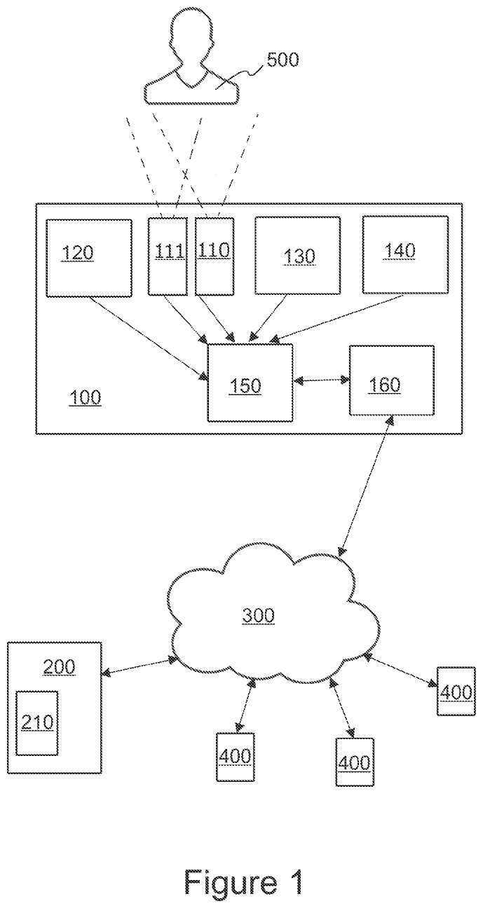

The present invention relates to a temperature monitoring system in order to provide monitoring body temperatures of individuals (500) like patient, baby, etc. in a remote and contactless manner. With reference to FIG. 1, the temperature monitoring system comprises a temperature monitoring device (100) in order to be positioned in the medium where said individual (500) exists. Said temperature monitoring device (100) comprises a thermal sensor (110) which faces towards the individual (500). The thermal sensor (110) measures the temperature values in the view area (FOV) and turns them into temperature measurements in matrix form. Such sensors are known in the art. These sensors sense the infrared radiation radiated due to the temperatures of objects and generate value/voltage accordingly.

The temperature monitoring device (100) also comprises a medium temperature sensor (120). The medium temperature sensor (120) generates a medium temperature value according to the temperature in the medium.

The temperature monitoring device (100) also comprises a humidity sensor (130). The humidity sensor (130) generates humidity measurements by measuring the humidity in the medium.

The temperature monitoring device (100) also comprises a distance sensor (140). Said distance sensor (140) generates distance measurements according to the distance of the individual (500) with respect to the distance sensor (140).

The temperature monitoring device (100) comprises a processor unit (150). Said processor unit (150) is connected to the distance sensor (140), the medium temperature sensor (120), the thermal sensor (110) and the humidity sensor (130). The processor unit (150) takes the measurement values from the sensors they are connected to. The processor unit (150) can be a microprocessor provided on a board. The processor unit (150) can have a processor and temporary and permanent memories for providing realization of predetermined processes by the processor. The processor unit (150) can be a processor unit (150) like Raspberry Pi used commercially.

The temperature monitoring device (100) also comprises a communication unit (160) associated with the processor unit (150) in a manner providing data exchange. Said communication unit (160) provides connection of the temperature measurement device to a communication network (300). The communication unit (160) can be connected to a communication network (300) by means of a modem, etc. (not illustrated in the figures) in a wired or wireless manner. Said communication network (300) is internet.

The temperature monitoring system also comprises a server (200) connected to the communication network (300). The temperature monitoring system moreover comprises mobile devices (400) configured to communicate with the server (200). Said mobile devices (400) can be a mobile phone, computer, tablet computer, etc. The mobile devices (400) can comprise an application for processing and displaying of the data coming from the server (200).

The temperature monitoring system, whose details can be given as above, functions as follows: The temperature monitoring device (100) is placed in a manner facing towards the location (for instance, bed) where the individual (500) like baby, patient, etc. exists. The thermal sensor (110) sends the temperature measurements in matrix form to the processor unit (150). The medium temperature sensor (120) sends the medium temperature measurements to the processor unit (150). The humidity sensor (130) sends the humidity measurements to the processor unit (150). The distance sensor (140) sends the distance measurements to the processor unit (150). The processor unit (150) provides sending these measurements to the processor by means of the communication unit (160). The server (200) selects one of temperature values in matrix form. The highest temperature value is selected by assuming that the highest temperature in the medium will be the body temperature of the individual (500). The server (200) records the temperature value, humidity measurement, distance measurement and medium temperature measurement values to a first database (210). It arranges the selected temperature value according to the humidity measurement values, the distance measurement values and the medium temperature measurement values, in other words, it calibrates the selected temperature value. As a result of this calibration, an individual (500) obtains the body temperature value. The individual (500) records the body temperature to the database together with the related measurements. Thus, all medium measurements and the individual (500) body temperature measurements are recorded. The server (200) also uses the pre-recorded measurement values during the calibration process. In other words, the calibration is continuously improved by realizing machine learning.

The server sends a warning message to the mobile devices (400) in case the measurement of body temperature of the individual (500) exceeds a predetermined first threshold value and/or in case the measurement of body temperature of the individual (500) is lower than a second threshold value. The server (200) can moreover send the instantaneous individual (500) body temperature or other measurements to the mobile devices (400) in order to be displayed. The first and the second threshold values are selected according to normal body temperature values of the individual (500).

The server moreover sends message to the mobile devices (400) indicating whether there is an excessively hot object in case the body temperature of the individual (500) exceeds a predetermined third threshold value. The server (200) sends a message, indicating that there is an excessively cold object in the medium, to the mobile device (400) in case the body temperature of the individual (500) is lower than a predetermined fourth threshold value. The first threshold values and the second threshold values are selected at values which may affect the body temperature of the individual (500) in an unfavorable manner much below and much above the normal body temperature of the individual (500).

The temperature monitoring device (100) also comprises a light sensor (111) for being directed to the patient. The light sensor (111) generates signal according to the light falling thereon. The light sensor (111) can be a thermal camera, camera, face recognition system, skin recognition system, etc. The processor unit (150) transfers the signals, received from the light sensor (111), to the server (200). The server (200) determines that the sensor is directed to a person in case the received signals meet the predetermined criteria. In case the sensor is a thermal camera, the server (200) receives the thermal image signal and can determine that the thermal image signal is directed to a human in case the thermal image is compliant to the predetermined criteria. In case the sensor is a camera, face recognition system, skin recognition system in a similar manner, it is detected whether the signal is compliant to predetermined criteria or not and it is determined whether the sensor is directed to a human or not. In case it is determined that the sensor is not directed to a human, an error signal is generated.

The protection scope of the present invention is set forth in the annexed claims and cannot be restricted to the illustrative disclosures given above, under the detailed description. It is because a person skilled in the relevant art can obviously produce similar embodiments under the light of the foregoing disclosures, without departing from the main principles of the present invention.

### REFERENCE NUMBERS

100 Temperature monitoring device
110 Thermal sensor
111 Light sensor
120 Medium temperature sensor
130 Humidity sensor
140 Distance sensor
150 Processor unit
160 Communication unit

200 Server
210 First database

300 Communication network

400 Mobile device

500 Individual

1. A temperature monitoring system for providing monitoring of the temperature of an individual in a contactless manner, comprising:
a thermal sensor configured to generate a plurality of temperature measurements in matrix form related to a measurement area where the thermal sensor is directed;
a distance sensor configured to face towards the measurement area and to generate a distance measurement in accordance with the distance of an individual, which exists in the measurement area, with respect to said distance sensor;
a processor unit connected to said thermal sensor and to said distance sensor, and configured to receive the plurality of temperature measurements and the distance measurement in matrix form as input; and
a communication unit connected to said processor unit and which provides communication of the processor unit with a server;
wherein:
said processor unit is configured to send the plurality of temperature measurements and the distance measurement to said server; and
said server is configured to select at least one of the temperature measurements in matrix form and to form an individual body temperature value by arranging the selected temperature measurement with respect to the distance measurement.

2. The temperature monitoring system according to claim 1, wherein the server is configured to send a warning message to at least one mobile device in case the formed individual body temperature value exceeds a predetermined first threshold value or in case the formed individual body temperature value is lower than a predetermined second threshold value.

3. The temperature monitoring system according to claim 1, further comprising a humidity sensor connected to the processor unit, the humidity sensor configured to realize a humidity measurement in the medium where the individual exists; and
wherein the processor unit is configured to send the humidity measurements to the server; and
wherein the server is configured to form the body temperature of the individual by arranging the selected temperature measurement.

4. The temperature monitoring system according to claim 3, further comprising a medium temperature sensor connected to the processor unit and configured to realize a medium temperature measurement in the medium where the individual exists, wherein the processor unit is configured to send the medium temperature measurement to the server, and wherein the server is configured to form the body temperature value of the individual by arranging the selected temperature measurement by taking into account the medium temperature measurements.

5. The temperature monitoring system according to claim 1, wherein the server is arranged to select the biggest among the temperature measurement in matrix form.

6. The temperature monitoring system according to claim 2, wherein:
the warning message, indicates that there is an excessively hot object in the medium in case the formed individual body temperature value exceeds a predetermined third threshold value; and/or
the warning message indicates that there is an excessively cold object in the medium in case the individual body temperature value is lower than a predetermined fourth threshold value.

7. The temperature monitoring system according to claim 1, wherein the server is configured to store the formed individual body temperatures in a first database.

8. The temperature monitoring system according to claim 4, wherein the server is configured to store in a first database the humidity measurement, medium temperature measurement, selected temperature measurement and individual body temperature values and to realize the next individual body temperature formation processes by taking into account also this database.

9. The temperature monitoring system according to claim 1, further comprising a light sensor configured to generate a signal according to the light received from the area where it is directed and to send this to the processor unit for being transferred to the server, wherein said server is configured to determine that the light sensor is directed to a human in case it is detected that the signals received from said light sensor, is compliant with a predetermined criteria.

10. The temperature monitoring system according to claim 9, wherein the server is configured to generate a warning signal in case the light sensor is not directed to a human.

11. The temperature monitoring system according to claim 9, wherein said light sensor is selected from the group consisting of a thermal camera, a color sensor, a face recognition system, and a skin recognition system.
